(19) United States
(12) Patent Application Publication
(10) Pub. No.: US 2024/0197545 A1
(43) Pub. Date: Jun. 20, 2024

(54) FEMININE HYGIENE PRODUCT WITH PROTRUSION

(71) Applicant: Emori LLC, Boston, MA (US)

(72) Inventor: Rehab Khawaga, Boston, MA (US)

(73) Assignee: Emori LLC, Boston, MA (US)

(21) Appl. No.: 18/287,066

(22) PCT Filed: Apr. 18, 2022
(86) PCT No.: PCT/US22/25196

§ 371 Date: Oct. 16, 2023

Related U.S. Application Data
(60) Provisional application No. 63/175,593, filed on Apr. 16, 2021.

(51) Int. Cl. A61F 13/472; A61F 13/475; A61F 13/84

(52) U.S. Cl. CPC A61F 13/47227; A61F 13/47263; A61F 13/4756; A61F 13/8405

(57) ABSTRACT

A feminine hygiene product that includes a top layer, a base layer, a protrusion formed in the top layer, a fluid absorbing section, and a reservoir section. The protrusion includes a primary channel, and the protrusion is configured to be inserted within an orifice of a user. The fluid absorbing section includes a plurality of fluid channels, and the plurality of fluid channels are configured to wick fluid away from the primary channel of the protrusion. The plurality of fluid channels have a gradient of absorbency, and a highest absorbency is distal from a proximal opening of the primary channel. The primary channel of the protrusion is positioned within the fluid absorbing section. The reservoir section is positioned between the top layer and the base layer. The reservoir section is positioned at a distal opening of the primary channel of the protrusion and comprises an absorbent material.

### CROSS-REFERENCE TO RELATED APPLICATION

This application claims priority to U.S. Provisional Application No. 63/175,593 filed on Apr. 16, 2021, the entire contents of which are incorporated herein by reference.

### BACKGROUND

### Field

The present disclosure is related to hygiene articles, specifically, feminine hygiene products with a non-woven absorbent material for collecting menstrual fluids, clots, and other bodily fluids. More particularly, this disclosure is directed to a feminine hygiene product with a protrusion configured for insertion into an orifice.

### Description of Related Art

Feminine hygiene products may be personal care products used by females during menstruation, after giving birth, or in response to vaginal discharge and other bodily functions related to other products used with the vulva and vagina. In some instances, a feminine hygiene product that is used during menstruation may be called a menstrual hygiene product. Feminine hygiene products may include sanitary napkins (e.g., sanitary towels, pads, such as sanitary pads, menstrual pads, etc.).

A sanitary napkin may be an absorbent item worn in a user's underwear when menstruating, bleeding after giving birth, recovering from gynecologic surgery, experiencing a miscarriage, and/or in any other situations where it may be necessary to absorb a flow of blood and/or discharge from the vagina. In some instances, a sanitary napkin may be a type of feminine hygiene product that is worn externally, unlike tampons and menstrual cups, which are worn inside the vagina.

### SUMMARY

The present disclosure relates generally to a feminine hygiene product with a protrusion configured for insertion into an orifice.

Further embodiments are set forth in the following numbered clauses:
Clause 1: A feminine hygiene product comprising: a top layer; a base layer; a protrusion formed in the top layer, the protrusion having a primary channel, wherein the protrusion is configured to be inserted within an orifice of a user; a fluid absorbing section, wherein the fluid absorbing section comprises a plurality of fluid channels, wherein the plurality of fluid channels are configured to wick fluid away from the primary channel of the protrusion; wherein the plurality of fluid channels have a gradient of absorbency, and the highest absorbency is distal from a proximal opening of the primary channel; wherein the primary channel of the protrusion is positioned within the fluid absorbing section; and a reservoir section positioned between the top layer and the base layer, wherein the reservoir section is positioned at a distal opening of the primary channel of the protrusion, and wherein the reservoir section comprises an absorbent material.
Clause 2: The feminine hygiene product of clause 1, wherein the reservoir section comprises a medicament.
Clause 3: The feminine hygiene product of clause 1 or 2, further comprising: a longitudinal ridge formed in the top layer extending lengthwise along the base.
Clause 4: The feminine hygiene product of any of clauses 1-3, further comprising: an exterior cover layer configured to fit over the top layer.
Clause 5: The feminine hygiene product of any of clauses 1-4, wherein the top layer is embedded with topical lotions, antibacterial lotions, or whitening agents.
Clause 6: The feminine hygiene product of any of clauses 1-5, wherein an outer peripheral edge of the top layer comprises an adhesive.
Clause 7: The feminine hygiene product of any of clauses 1-6, wherein an anterior crest of the longitudinal ridge comprises an absorbent material.
Clause 8: The feminine hygiene product of any of clauses 1-7, wherein the top layer comprises a medicament.
Clause 9: The feminine hygiene product of any of clauses 1-8, wherein the fluid absorbing section comprises a non-woven, low-swelling, absorbent polymer.
Clause 10: The feminine hygiene product of any of clauses 1-9, wherein the plurality of fluid channels comprise a gradient ratio of cellulose pulp to absorbent polymer.
Clause 11: The feminine hygiene product of any of clauses 1-10, wherein the protrusion of the top layer comprises a medicament.
Clause 12: The feminine hygiene product of any of clauses 1-11, wherein the absorbent material of the reservoir section is porous.
Clause 13: The feminine hygiene product of any of clauses 1-12, wherein the reservoir section further comprises a cavity.
Clause 14: The feminine hygiene product of any of clauses 1-13, wherein the reservoir section further comprises: a first layer comprising the medicament; and a second layer comprising a fibrous mesh.
Clause 15: The feminine hygiene product of any of clauses 1-14, wherein the reservoir section further comprises: a third layer comprising a plurality of absorbent composite polymers.
Clause 16: The feminine hygiene product of any of clauses 1-15, wherein the first layer comprises a concentration of the medicament, the second layer comprises a concentration of the medicament, and the third layer comprises a concentration of the medicament, wherein the concentration of the medicament of the first layer is higher than the concentration of the medicament of the second layer or the concentration of the medicament of the third layer.
Clause 17: The feminine hygiene product of any of clauses 1-16, further comprising: a heating device.
Clause 18: A feminine hygiene product comprising: a top layer; a base layer; a protrusion formed in the top layer, the protrusion having a primary channel, wherein the protrusion is configured to be inserted within an orifice of a user; a fluid absorbing section, wherein the fluid absorbing section comprises a plurality of fluid channels; wherein the plurality of fluid channels are configured to wick fluid away from the primary channel of the protrusion; wherein each of the plurality of fluid channels comprises a material having an absorption metric; wherein the plurality of fluid channels have a gradient of absorbency; wherein a first fluid channel of the plurality of fluid channels has a material having a first absorption metric; wherein a second fluid channel of the plurality of fluid channels has a material having a second absorption metric; wherein the first absorption metric is greater than the second absorption metric; wherein the first fluid channel is adjacent to the second fluid channel; and wherein the primary channel of the protrusion is positioned within the fluid absorbing section; and a reservoir section positioned between the top layer and the base layer, wherein the reservoir section is positioned at a distal opening of the primary channel of the protrusion, and wherein the reservoir section comprises an absorbent material.
Clause 19: The feminine hygiene product of clause 18, wherein the reservoir section comprises a medicament.
Clause 20: The feminine hygiene product of clause 18 or 19, further comprising: a longitudinal ridge formed in the top layer extending lengthwise along the base layer.
Clause 21: The feminine hygiene product of any of clauses 18-20, further comprising: an exterior cover layer configured to fit over the top layer.
Clause 22: The feminine hygiene product of any of clauses 18-21, wherein the top layer is embedded with topical lotions, antibacterial lotions, or whitening agents.
Clause 23: The feminine hygiene product of any of clauses 18-22, wherein an outer peripheral edge of the top layer comprises an adhesive.
Clause 24: The feminine hygiene product of any of clauses 18-23, wherein an anterior crest of the longitudinal ridge comprises an absorbent material.
Clause 25: The feminine hygiene product of any of clauses 18-24, wherein the top layer comprises a medicament.
Clause 26: The feminine hygiene product of any of clauses 18-25, wherein the fluid absorbing section comprises a non-woven, low-swelling, absorbent polymer.
Clause 27: The feminine hygiene product of any of clauses 18-26, wherein the plurality of fluid channels comprise a gradient ratio of cellulose pulp to absorbent polymer.
Clause 28: The feminine hygiene product of any of clauses 18-27, wherein the protrusion of the top layer comprises a medicament.
Clause 29: The feminine hygiene product of any of clauses 18-28, wherein the absorbent material of the reservoir section is porous.
Clause 30: The feminine hygiene product of any of clauses 18-29, wherein the reservoir section further comprises a cavity.
Clause 31: The feminine hygiene product of any of clauses 18-30, wherein the reservoir section further comprises: a first layer comprising the medicament; and a second layer comprising a fibrous mesh.
Clause 32: The feminine hygiene product of any of clauses 18-31, wherein the reservoir section further comprises: a third layer comprising a plurality of absorbent composite polymers.
Clause 33: The feminine hygiene product of any of clauses 18-32, wherein the first layer comprises a concentration of the medicament, the second layer comprises a concentration of the medicament, and the third layer comprises a concentration of the medicament, wherein the concentration of the medicament of the first layer is higher than the concentration of the medicament of the second layer or the concentration of the medicament of the third layer.
Clause 34: The feminine hygiene product of any of clauses 18-33, further comprising: a heating device.
Clause 35: A feminine hygiene product comprising: a top layer; a base layer; a protrusion formed in the top layer, the protrusion having a primary channel, wherein the protrusion is configured to be inserted within an orifice of a user; a fluid absorbing section, wherein the fluid absorbing section comprises a plurality of fluid channels, wherein the plurality of fluid channels are configured to wick fluid away from the primary channel of the protrusion; a reservoir section positioned between the top layer and the base layer, wherein the reservoir section is positioned at a distal opening of the primary channel of the protrusion, and wherein the reservoir section comprises an absorbent material; and a heating device, wherein the heating device comprises an aperture, and wherein the aperture is configured to fit over the protrusion formed in the top layer.
Clause 36: The feminine hygiene product of clause 35, wherein the reservoir section comprises a medicament.
Clause 37: The feminine hygiene product of clause 35 or 36, further comprising: a longitudinal ridge formed in the top layer extending lengthwise along the base.
Clause 38: The feminine hygiene product of any of clauses 35-37, wherein an outer peripheral edge of the top layer comprises an adhesive.
Clause 39: The feminine hygiene product of any of clauses 35-38, wherein an anterior crest of the longitudinal ridge comprises an absorbent material.
Clause 40: The feminine hygiene product of any of clauses 35-39, wherein the fluid absorbing section comprises a non-woven, low-swelling, absorbent polymer.
Clause 41: The feminine hygiene product of any of clauses 35-40, wherein the plurality of fluid channels comprise a gradient ratio of cellulose pulp to absorbent polymer.
Clause 42: The feminine hygiene product of any of clauses 35-41, wherein the protrusion of the top layer comprises a medicament.
Clause 43: The feminine hygiene product of any of clauses 35-42, wherein the absorbent material of the reservoir section is porous.
Clause 44: The feminine hygiene product of any of clauses 35-43, wherein the reservoir section further comprises a cavity.
Clause 45: The feminine hygiene product of any of clauses 35-44, wherein the reservoir section further comprises: a first layer comprising the medicament; and a second layer comprising a fibrous mesh.
Clause 46: The feminine hygiene product of any of clauses 35-45, wherein the reservoir section further comprises: a third layer comprising a plurality of absorbent composite polymers.
Clause 47: The feminine hygiene product of any of clauses 35-46, wherein the first layer comprises a concentration of the medicament, the second layer comprises a concentration of the medicament, and the third layer comprises a concentration of the medicament, wherein the concentration of the medicament of the first layer is higher than the concentration of the medicament of the second layer or the concentration of the medicament of the third layer.
Clause 48: The feminine hygiene product of any of clauses 35-47, wherein the heating device comprises two wings, wherein each of the two wings comprises a metal strip, and wherein the metal strip is configured to be bent to support a positioning of the heating device.
Clause 49: The feminine hygiene product of any of clauses 35-48, wherein the two wings are configured to be fastened together.
Clause 50: The feminine hygiene product of any of clauses 35-49, wherein the plurality of fluid channels have a gradient of absorbency, and the highest absorbency is distal from a proximal opening of the primary channel; and wherein the primary channel of the protrusion is positioned within the fluid absorbing section.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present disclosure. As used in the specification and the claims, the singular form of “a,” “an,” and “the” include plural referents unless the context clearly dictates otherwise.

### BRIEF DESCRIPTION OF THE DRAWINGS

FIG. 1 is a diagram of a perspective view of a non-limiting embodiment of a feminine hygiene product;

FIG. 2 is a diagram of a top down view of the feminine hygiene product shown in FIG. 1;

FIGS. 3-5 are diagrams of a cross section along line A-A of the feminine hygiene product shown in FIG. 2;

FIG. 6 is a diagram of a cross section of a reservoir section of the feminine hygiene product shown in FIG. 1;

FIG. 7 is a diagram of a perspective view of a non-limiting embodiment of a feminine hygiene product with an exterior cover layer;

FIG. 8 is a diagram of a perspective view of a non-limiting embodiment of a feminine hygiene product with a heating device; and

FIGS. 9-11 are diagrams of a perspective view of non-limiting embodiments of fluid absorbency sections of a feminine hygiene product.

### DETAILED DESCRIPTION

Various examples and embodiments of the subject matter disclosed are possible and will be apparent to a person of ordinary skill in the art, given the benefit of this disclosure. In this disclosure, reference to “some embodiments,” “certain embodiments,” “certain exemplary embodiments,” and similar phrases each means that those embodiments are non-limiting examples of the inventive subject matter, and there may be alternative embodiments which are not excluded.

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments. Hence, specific dimensions and other physical characteristics related to the embodiments disclosed herein are not to be considered as limiting.

Also, as used herein, the articles “a” and “an” are intended to include one or more items, and may be used interchangeably with “one or more” and “at least one.” As used in the specification and the claims, the singular form of “a,” “an,” and “the” include plural referents, unless the context clearly dictates otherwise. Additionally, as used herein, the terms “set” and “group” are intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

It is desirable in the art to create a feminine hygiene product which is lightweight, comfortable, and leak-proof. Provided is a feminine hygiene product with a protrusion configured for insertion into an orifice. Embodiments of the present disclosure may include a top layer; a base layer; a protrusion formed in the top layer, the protrusion having a primary channel, wherein the protrusion is configured to be inserted within an orifice of a user; a fluid absorbing section, wherein the fluid absorbing section comprises a plurality of fluid channels, wherein the plurality of fluid channels are configured to wick fluid away from the primary channel of the protrusion; wherein the plurality of fluid channels have a gradient of absorbency, and a highest absorbency (e.g., an area of highest absorbency of a fluid channel, an area of highest absorbency of each fluid channel, a fluid channel having a material such that the fluid channel has a highest absorbency, etc.) is distal from a proximal opening of the primary channel; wherein the primary channel of the protrusion is positioned within the fluid absorbing section; and a reservoir section positioned between the top layer and the base layer, wherein the reservoir section is positioned at a distal opening of the primary channel of the protrusion, wherein the reservoir section comprises an absorbent material.

The feminine hygiene product may have a protrusion which is made of several layers of veiled lateral absorbing channels. In some non-limiting embodiments, the body-facing surface, or top layer, may include a liquid impervious top layer to cover the inner absorbing layers. The protrusion may be hollow (e.g., have a three-dimensional shape) and placed centrally within the feminine hygiene product and act to wick the fluid and/or agglomerations (e.g., clots) to the inner area of the product as they pass through a primary channel and along absorbent fluid channels. The reservoir section may be designed for collection of agglomerations and may contain a medicament to allow for disintegration of the agglomerations and, therefore, greater absorbance. In some non-limiting embodiments, feminine hygiene product may be visually effective. For example, current products may include top covers which allow the user to see through the cover and to view the fluids, which may result in an unpleasant experience for the wearer. The present disclosure a feminine hygiene product with a top layer (e.g., an opaque top layer) suitable to hide an appearance of the collected materials below.

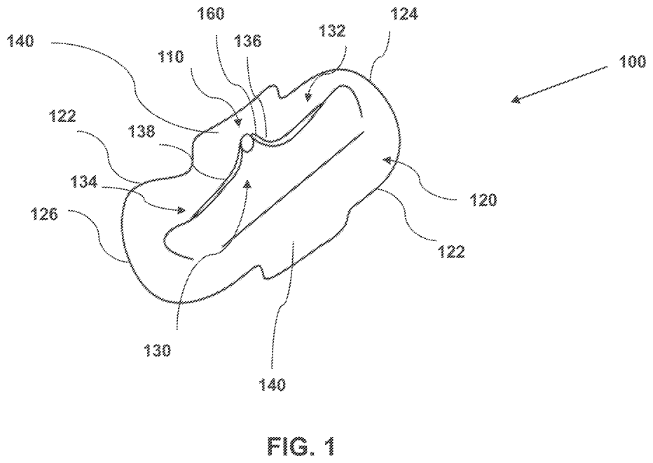

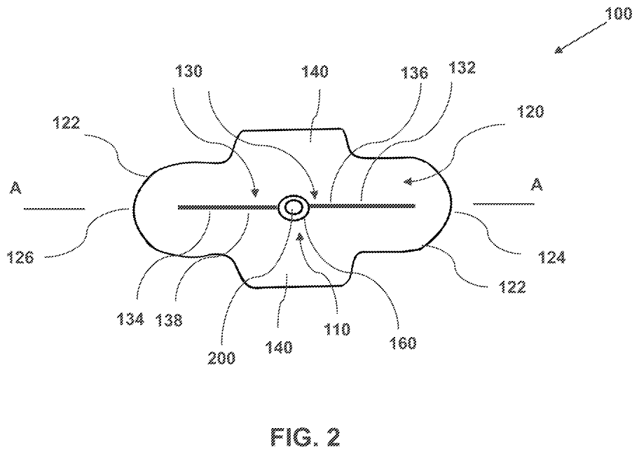

Referring now to FIGS. 1 and 2, FIGS. 1 and 2 are diagrams of a non-limiting embodiment of feminine hygiene product 100. As shown in FIGS. 1 and 2, feminine hygiene product 100 may include protrusion 110, top layer 120, and longitudinal ridge 130. As further shown in FIGS. 1 and 2, longitudinal ridge 130 may include anterior protrusion 132 and posterior protrusion 134, and anterior protrusion 132 may be formed with anterior crest 136 at a top of longitudinal ridge 130, and posterior protrusion 134 may be formed with posterior crest 138 at a top of longitudinal ridge 130. In some non-limiting embodiments, an outer perimeter of feminine hygiene product 100 may be defined by outer peripheral edge 122. In some non-limiting embodiments, feminine hygiene product 100 may include an absorbent material in any of the components of feminine hygiene product 100, such an absorbent material is described throughout and may include a super absorbent polymer (SAP) and/or bacterial cellulose.

In some non-limiting embodiments, longitudinal ridge 130 may extend between anterior end 124 and posterior end 126. For example, longitudinal ridge 130 may begin at anterior end 124 and extend to posterior end 126. In another example, a beginning of longitudinal ridge 130 may be offset from anterior end 124 and/or posterior end 126.

In some non-limiting embodiments, top layer 120 may be formed as the outer most layer of feminine hygiene product 100. For example, top layer 120 may be configured to be a layer that is in contact with the user (e.g., in contact with an area surrounding an orifice of the user and/or area inside the orifice of the user). In some non-limiting embodiments, longitudinal ridge 130 (e.g., anterior protrusion 132 and/or posterior protrusion 134 of longitudinal ridge 130) and/or protrusion 110 may be formed on top layer 120.

In some non-limiting embodiments, top layer 120 may include one or more materials. In some non-limiting embodiments, top layer 120 may be made of a soft, absorbent and/or non-absorbent material. For example, top layer 120 may be made of a porous layer of hydrophobic polyester and/or hydrophobic polypropylene (e.g., propylene) fibers. In some non-limiting embodiments, top layer 120 may include a foam (e.g., natural latex foam), polyurethane, and/or bacterial cellulose.

In some non-limiting embodiments, top layer 120 may include a non-woven material. The non-woven material may be a spunmelt non-woven material, where fibers of the material are bonded together by chemical, mechanical, heat, and/or solvent treatments. In some non-limiting embodiments, top layer 120 may include a non-woven material formed through an air-bonding, a thermal bonding, and/or a hydroentanglement process. In some non-limiting embodiments, top layer 120 may include fibers, such as polypropylene, polyethylene, rayon, polyester(s), polylactic acid, and/or bio-component fibers. Fibers of top layer 120 may also be natural fibers, such as cotton, wood pulp, hemp, and/or flax.

In some non-limiting embodiments, top layer 120 may include other materials, such as films which may be made from polyethylene, polypropylene. In some non-limiting embodiments, films in top layer 120 may include apertures to control a pore size going into feminine hygiene product 100 and limit fluid coming back out of feminine hygiene product 100.

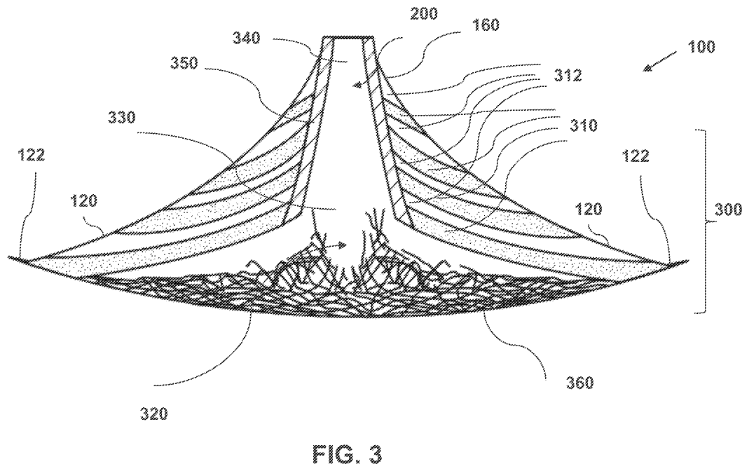

In some non-limiting embodiments, the entirely of or portions of top layer 120 may be made impervious (e.g., impermeable) to fluid. For example, top layer 120 may include a material that is impervious to fluid. Additionally or alternatively, one or more sections of top layer 120 may be pervious (e.g., permeable) to fluid. In some non-limiting embodiments, one or more sections of top layer 120 may be impervious to fluid to allow only protrusion 110 and/or primary channel 200 to channel some or all of the liquid into a fluid absorbing section (e.g., fluid absorbing section 300 shown in FIG. 3) and/or a plurality of fluid channels (e.g., first plurality of fluid channels 310 and/or second plurality of fluid channels 312). In some non-limiting embodiments, top layer 120 may allow fluid to pass through pores of top layer 120 while remaining dry (e.g., dry to the touch of a user). In some non-limiting embodiments, top layer 120 may be hydrophobic and/or hydrophilic. In some non-limiting embodiments, top layer 120 may be hydrophobic while also being configured to retain some amount of liquid. For example, top layer 120 may retain some amount of liquid in the interstices of the pores of top layer 120.

In some non-limiting embodiments, top layer 120 may include a polypropylene non-woven sheet. In some non-limiting embodiments, the polypropylene non-woven sheet of top layer 120 may be inserted between other materials of top layer 120 by using a hot melt process and/or a heat pressure method. In some non-limiting embodiments, top layer 120 may include a lightweight, cloth-like non-woven laminate. In some non-limiting embodiments, top layer 120 may include a nanofiber web that may be pre-moistened.

In some non-limiting embodiments, top layer 120 may be embedded with a substance, such as particulates and/or medicaments (e.g., medications). For example, top layer 120 may include a medicament. In some non-limiting embodiments, the substance may include a pelvic health medication, a heating agent, a hair growth delay substance, particulates that promote relaxation and muscle tension relief, and/or the like. In some non-limiting embodiments, the substance may include a material for heating or soothing the user, such as capsaicin, sodium acetate, sodium salicylate, and/or other materials which are known to crystallize upon exposure to air, thereby producing a mild exothermic reaction. Additionally or alternatively, the substance may include an ointment (e.g., salve, balm, lotion, etc.). In some non-limiting embodiments, the substance may include aloe vera, almond oil, vitamin E, vitamin D, an antibacterial lotion (e.g., tertiary ammonia, silver salt containing compounds, etc.), and/or an intimate area whitening agent. For example, top layer 120 may be embedded with topical lotions, antibacterial lotions, and/or whitening agents.

In some non-limiting embodiments, top layer 120 may include adhesive material. For example, adhesive materials can also be incorporated in top layer 120 for better positioning (e.g., relative to the user) and stability of feminine hygiene product 100. Adhesives may include pressure-sensitive adhesives and/or a drape adhesion material, such as a gecko-inspired adhesive, Geckskin®.

In some non-limiting embodiments, feminine hygiene product 100 may include outer peripheral edge 122 formed in top layer 120. In some non-limiting embodiments, outer peripheral edge 122 may be a peripheral edge of feminine hygiene product 100. In some non-limiting embodiments, outer peripheral edge 122 may completely surround feminine hygiene product 100. In some non-limiting embodiments, top layer 120 may include an adhesive positioned along outer peripheral edge 122 of feminine hygiene product 100, which may provide a way for direct attachment of feminine hygiene product 100 to the user. This may enable the user to use (e.g., wear) feminine hygiene product 100 without use of undergarments.

In some non-limiting embodiments, feminine hygiene product 100 may include a plurality of wings. For example, feminine hygiene product 100 may include wings 140. In some non-limiting embodiments, wings 140 may be formed in top layer 120. In some non-limiting embodiments, wings 140 may be a flexible material. In some non-limiting embodiments, wings 140 may be configured to be folded and/or bent around a user's underwear to provide security and/or stability of feminine hygiene product 100. In some non-limiting embodiments, wings 140 may include an adhesive material. For example, the bottom portion of wings 140 may include an adhesive material, such that the wings may adhere to the surface of a user's underwear to provide security and/or stability of feminine hygiene product 100.

In some non-limiting embodiments, feminine hygiene product 100 may include longitudinal ridge 130 formed in top layer 120. In some non-limiting embodiments, longitudinal ridge 130 may extend longitudinally lengthwise along feminine hygiene product 100. In some non-limiting embodiments, longitudinal ridge 130 may extend from anterior end 124 to posterior end 126 of feminine hygiene product 100.

In some non-limiting embodiments, anterior end 124 of feminine hygiene product 100 may be configured to be positioned toward the front-end of the user. In some non-limiting embodiments, posterior end 126 of feminine hygiene product 100 may be configured to be positioned toward the back-end of the user. For example, anterior end 124 may be configured to be positioned close to a labia of the user and/or posterior end 126 may be positioned close to the buttocks of the user.

In some non-limiting embodiments, longitudinal ridge 130 may include one or more protrusions. In some non-limiting embodiments, longitudinal ridge 130 may include anterior protrusion 132 and/or posterior protrusion 134. For example, anterior protrusion 132 may be formed at the anterior end of longitudinal ridge 130 and/or posterior protrusion 134 may be formed at the posterior end of longitudinal ridge 130. In some non-limiting embodiments, anterior protrusion 132 and/or posterior protrusion 134 may be sized and configured to hold feminine hygiene product 100 against the body of the user. For example, anterior protrusion 132 may be sized and configured to be positioned within a labia of the user and/or posterior protrusion 134 may be sized and configured to be positioned within the buttocks of the user.

In some non-limiting embodiments, anterior protrusion 132 of longitudinal ridge 130 may include an absorbent tip. For example, anterior protrusion 132 may include an absorbent tip positioned laterally along anterior crest 136 of anterior protrusion 132 to capture possible leakage. In some non-limiting embodiments, posterior protrusion 134 of longitudinal ridge 130 may include an absorbent tip. For example, posterior protrusion 134 may include an absorbent tip positioned laterally along posterior crest 138 of posterior protrusion 134 to capture possible leakage.

In some non-limiting embodiments, longitudinal ridge 130 may extend from a base layer (e.g., base layer 360 shown in FIG. 3). In some non-limiting embodiments, longitudinal ridge 130 may extend lengthwise along the base layer of feminine hygiene product 100.

In some non-limiting embodiments, feminine hygiene product 100 may include protrusion 110 formed in top layer 120. In some non-limiting embodiments, protrusion 110 may be sized and/or configured to be inserted within an orifice of a user (e.g., a wearer of feminine hygiene product 100). In some non-limiting embodiments, protrusion 110 may have a radius. In some non-limiting embodiments, protrusion 110 may include a material that is configured to maintain a level of stiffness when inserted into an orifice of a user. For example, protrusion 110 may include a material that is stiff enough to maintain an open pore structure of protrusion 110 but also be flexible and/or resilient to provide comfort to the user.

In some non-limiting embodiments, protrusion 110 may include a foam material. For example, protrusion 110 may include foam material sculpted or molded into a round or elongated shape. The foam material may be open-celled. In some non-limiting embodiments, protrusion 110 may have a vertical thickness. For example, protrusion 110 may include a non-woven foam material that is sculpted or molded into a round or elongated shape of a desired vertical thickness.

In some non-limiting embodiments, protrusion 110 may include a hi-loft non-woven material such as polyester. Additionally or alternatively, protrusion 110 may include bi-component fibers such as polyethylene and polyester or polypropylene and polyester. Additionally or alternatively, protrusion 110 may include co-polyesters. In some non-limiting embodiments, a density of the material of protrusion 110 may range from 0.01 grams per cubic centimeter (g/cc) to 0.05 g/cc.

In some non-limiting embodiments, protrusion 110 may include outer surface 160. In some non-limiting embodiments, protrusion 110 may be formed along longitudinal ridge 130. In some non-limiting embodiments, protrusion 110 may be formed between anterior protrusion 132 and posterior protrusion 134.

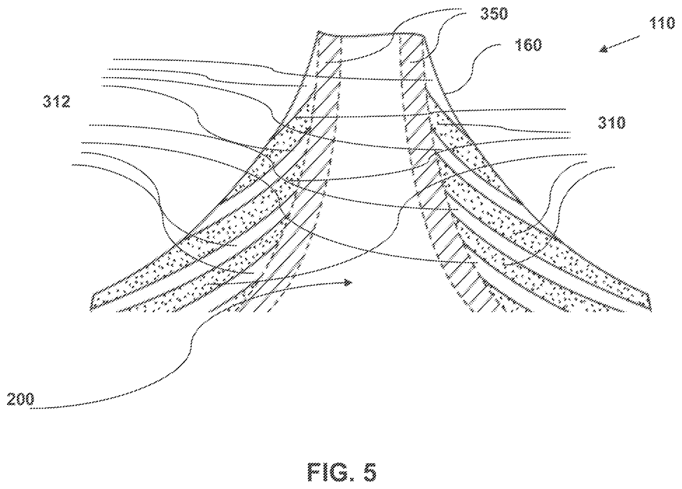

As further shown in FIG. 2, primary channel 200 may be formed within protrusion 110. In some non-limiting embodiments, primary channel 200 may be formed as part of protrusion 110. In some non-limiting embodiments, fluids may descend from the user and flow through primary channel 200. In some non-limiting embodiments, primary channel 200 may be configured to allow fluid to flow into an interior (e.g., as seen in FIGS. 3-6) of feminine hygiene product 100. For example, fluids from the user may flow into an interior of feminine hygiene product 100 via primary channel 200. In some non-limiting embodiments or aspects, primary channel 200 may be a hollow tube-like structure or hole.

In some non-limiting embodiments, primary channel 200 may be formed by rolling a material around a mandrel and sewing the material with a seam. In some non-limiting embodiments, primary channel 200 may be formed via a molding process. For example, primary channel 200 may be formed by being molded into protrusion 110. In some non-limiting embodiments, primary channel 200 may be formed by cutting out the structure of the primary channel 200 from a layer of non-woven material.

In some non-limiting embodiments, protrusion 110 and/or primary channel 200 may include outer surface 160. In some non-limiting embodiments, outer surface 160 may include a porous material. In some non-limiting embodiments, outer surface 160 may include a dressing, such as a hydrogel dressing and/or a gauze dressing. In some non-limiting embodiments, the dressing may be embedded with a medicament, such as a wound healing agent that may be useful for a user recovering from delivery of a child. In some non-limiting embodiments, outer surface 160 may be embedded with a substance, such as a particulate or a medication. In some non-limiting embodiments, the substance may include heating agents, pelvic health agents, hair delay substances, and/or particulates that promote relaxation and muscle tension relief.

Referring now to FIGS. 3-5, FIGS. 3-5 are diagrams of a cross section along line A-A of feminine hygiene product 100 (e.g., line A-A shown in FIG. 2). As shown in FIG. 3, feminine hygiene product 100 may include fluid absorbing section 300, reservoir section 320, and/or base layer 360. In some non-limiting embodiments, fluid absorbing section 300 may include a plurality of fluid channels that are designed to carry fluid away from primary channel 200 as the fluid travels down primary channel 200. As shown in FIGS. 3-5, fluid absorbing section 300 may include fluid channels 310, 312. In some non-limiting embodiments, one or more channels of first plurality of fluid channels 310 may include a material (e.g., an absorbing material) that is configured to carry fluid away from primary channel 200. Additionally or alternatively, one or more channels of second plurality of fluid channels 312 may be hollow channels (e.g., devoid of material) that are configured to carry fluid away from primary channel 200 and/or allow fluid to flow to one or more channels of first plurality of fluid channels 310. In some non-limiting embodiments, one or more channels of second plurality of fluid channels 312 may include a material (e.g., an absorbing material) that is configured to carry fluid away from primary channel 200. In some non-limiting embodiments, one or more channels of first plurality of fluid channels 310 and/or one or more channels of second plurality of fluid channels 312 may have a three-dimensional shape (e.g., a tube-like structure, such as a cylinder) or a two-dimensional shape (e.g., a flat or approximately flat structure that may be formed by embossing). In some non-limiting embodiments, the material of second plurality of fluid channels 312 may be the same as or similar to the material of first plurality of fluid channels 312.

In some non-limiting embodiments, primary channel 200 may include distal opening 330, proximal opening 340, and/or primary channel lining 350. In some non-limiting embodiments, primary channel 200 of protrusion 110 may be positioned within fluid absorbing section 300. In some non-limiting embodiments, primary channel 200 may include proximal opening 340 formed near or as part of protrusion 110. In some non-limiting embodiments, primary channel 200 may include distal opening 330 formed near or as part of reservoir section 320.

In some non-limiting embodiments, fluid absorbing section 300 may include a non-woven, low-swelling, absorbent material. For example, fluid absorbing section 300 may include a non-woven, low-swelling, absorbent polymer. As shown in FIGS. 3-5, fluid absorbing section 300 may include a first plurality of fluid channels 310 and/or a second plurality of fluid channels 312.

In some non-limiting embodiments, the plurality of fluid channels 310, 312 may be positioned along sides of fluid absorbing section 300. In some non-limiting embodiments, the first plurality of fluid channels 310 may be configured to direct (e.g., based on wicking) fluid away from primary channel 200 of protrusion 110, such that fluid is absorbed by a material of the first plurality of fluid channels 310 when the fluid descends in primary channel 200. In some non-limiting embodiments, the first plurality of fluid channels 310 and/or the second plurality of fluid channels 312 may have a gradient of absorbency. In some non-limiting embodiments, the first plurality of fluid channels 310 and/or the second plurality of fluid channels 312 may include a gradient ratio of materials. For example, the first plurality of fluid channels 310 and/or the second plurality of fluid channels 312 may include a gradient ratio of cellulose pulp to absorbent polymer. In some non-limiting embodiments, the first plurality of fluid channels 310 may be formed of a first material and the second plurality of fluid channels 312 may be formed of a second material. In some non-limiting embodiments, the first plurality of fluid channels 310 and/or the second plurality of fluid channels 312 may have a gradient of absorbency where an area of highest absorbency (e.g., an area of highest absorbency of a fluid channel, an area of highest absorbency of each fluid channel, a fluid channel of the plurality of fluid channels having a material such that the fluid channel has a highest absorbency, etc.) is distal from primary channel 200. For example the first plurality of fluid channels 310 and/or the second plurality of fluid channels 312 may have a gradient of absorbency where a highest absorbency is distal from proximal opening 340 of primary channel 200.

In some non-limiting embodiments, the plurality of fluid channels 310, 312 of fluid absorbing section may be layered alternating between the first material of the first plurality of fluid channels 310 and the second material of the second plurality of fluid channels 312. In some non-limiting embodiments, the gradient of absorbency of the plurality of fluid channels 310, 312 may be based on an absorbency of the materials used to form the plurality of fluid channels 310, 312. In some non-limiting embodiments, the materials used to form the plurality of fluid channels 310, 312 may have a first absorbency at or near proximal opening 340 of primary channel 200 and a second absorbency at or near outer peripheral edge 122 and/or top layer 120, where the first absorbency of the plurality of fluid channels 310, 312 is lower than the second absorbency of the plurality of fluid channels 310, 312. In some non-limiting embodiments, fluid channels 310, 312 positioned near distal opening 330 may have a higher absorbency than the plurality of fluid channels 310, 312 positioned near proximal opening 340, or vice versa.

As further shown in FIGS. 3-5, primary channel lining 350 may extend from a top of proximal opening 340 to a base of distal opening 330. In some non-limiting embodiments, primary channel lining 350 may line an interior of primary channel 200. In some non-limiting embodiments, primary channel lining 350 may include an absorbent and/or a non-absorbent material. In some non-limiting embodiments, primary channel lining 350 may have a first absorbency metric and a second absorbency metric. For example, a first portion of primary channel lining 350 positioned near proximal opening 340 of primary channel 200 may have a first absorbency metric, and a second portion of primary channel lining 350 positioned near distal opening 330 of primary channel 200 may have a second absorbency metric. In some non-limiting embodiments, the first absorbency metric of primary channel lining 350 may be lower than the second absorbency metric of primary channel lining 350.

In some non-limiting embodiments, fluid absorbing section 300 may include reservoir section 320. In some non-limiting embodiments, reservoir section 320 may be sized and configured to accept and/or absorb agglomerations (e.g., clots, solidified fluids, tissue etc.) that pass through primary channel 200. For example, reservoir section 320 may be configured to accept and/or absorb agglomerations that pass through primary channel 200 without flowing through the plurality of fluid channels 310, 312.

In some non-limiting embodiments, reservoir section 320 may be positioned between top layer 120 and base layer 360. In some non-limiting embodiments, reservoir section 320 may be positioned at distal opening 330 of primary channel 200 of protrusion 110. In some non-limiting embodiments, the plurality of fluid channels 310, 312 may extend to reservoir section 320. In some non-limiting embodiments, reservoir section 320 may include an absorbent material. In some non-limiting embodiments, the absorbent material of reservoir section 320 may be porous. In some non-limiting embodiments, reservoir section 320 may include a medicament. For example, reservoir section 320 may be embedded with a medicament. The medicament may comprise thrombolytic drugs which aid in resolution (e.g., breakdown) of agglomerations, such as clots. The resolved agglomerations may become entrapped within reservoir section 320 via a fibrous medium, such as a super absorbent fabric layer. In some non-limiting embodiments, distal opening 330 may be filled with highly efficient absorbents (e.g., absorbers), such as a super absorbent polymer (SAP) and/or bacterial cellulose.

In some non-limiting embodiments, top layer 120 of feminine hygiene product 100 may cover fluid absorbing section 300. For example, top layer 120 may be made of a material wherein the absorbed fluids and clots are wicked to the inside, specifically, to fluid absorbing section 300. In some non-limiting embodiments, the material of top layer 120 may allow the absorbed fluids and/or agglomerations to be blocked visually and/or physically (e.g., from contact with the skin of a user). In some non-limiting embodiments, top layer 120 may be made of a material with high opacity and/or may not include interstices to allow fluid to pass except for an aperture within top layer 120 to allow for fluid to pass through primary channel 200 of protrusion 110.

In some non-limiting embodiments, protrusion 110 of feminine hygiene product 100 may be made of absorbent polymer (e.g., non-woven, low-swelling, and absorbent polymer). For example, an outside and/or an inside of protrusion 110 of feminine hygiene product 100 may be made of absorbent polymer. In some non-limiting embodiments, protrusion 110 of feminine hygiene product 100 may include a ring like structure at a top of protrusion 110 with inner and outer parts made of absorbent polymer. In this way, the ring-like structure may help in the attachment of feminine hygiene product 100 and also provide a leak-free experience. In some non-limiting embodiments, the use of a low-swelling polymer may allow more time for fluids to be distributed within the plurality of fluid channels 310, 312 of feminine hygiene product 100. This results in less saturation of protrusion 110, reducing uncomfortable feelings by the user, including a feeling of wetness. In some non-limiting embodiments, protrusion 110 may be configured to quickly pass fluid through to fluid channels 310, while at the same time restricting passage of fluid in a reverse direction. In some non-limiting embodiments, restricting passage of fluid in the reverse direction may be accomplished by a gradient of absorbency and/or use of an effective rewetting agent within primary channel 200.

In some non-limiting embodiments, a gradient of absorbency within fluid channels 310 may be optimized based on a specific ratio of cellulose pulp fluff to a super absorbent polymer. In some non-limiting embodiments, the porosity of the nonwoven material within primary channel 200 may result in a gradient of absorbency which may allow the fluid to be adequately wicked towards distal opening 330 of fluid absorbing section 300. In this way, the distal ends of fluid channels 310 (e.g., the ends of fluid channels 310 positioned near top layer 120 and/or distal opening 330) may become saturated before the proximal ends of fluid channels 310 (e.g., the ends of fluid channels 310 positioned near primary channel 200) become saturated. In this way, agglomerations may descend by gravity and an opening of primary channel 200 may become wider to allow larger agglomerations to enter reservoir section 320.

In some non-limiting embodiments, fluid absorbing section 300 may include a plurality of fluid channels 310. The plurality of fluid channels 310 may be configured to wick fluid away from primary channel 200 of protrusion 110. In some non-limiting embodiments, a first fluid channel of the plurality of fluid channels 310 may be adjacent to a second fluid channel of the plurality of fluid channels 310. In some non-limiting embodiments, each of the plurality of fluid channels 310 may include a material having an absorption metric (e.g., grams per cubic centimeter). In some non-limiting embodiments, the first fluid channel of the plurality of fluid channels 310 may have a first absorption metric. In some non-limiting embodiments, the second fluid channel of the plurality of fluid channels 310 may have a second absorption metric. In some non-limiting embodiments, the first absorption metric of the first fluid channel may be greater than or less than the second absorption metric of the second fluid channel.

In some non-limiting embodiments, the absorption metric of a fluid channel of the plurality of fluid channels 310 may vary. In some non-limiting embodiments, a single fluid channel may include at least a first section and a second section. The first section of the fluid channel may be closer to primary channel 200 than the second section of the fluid channel. In some non-limiting embodiments, the first section of the fluid channel may have a lower absorbency than the second section of the fluid channel.

In some non-limiting embodiments, reservoir section 320 may be sized and configured to collect agglomerations that pass through primary channel 200. In some non-limiting embodiments, reservoir section 320 may be made of a fibrous and/or non-woven mesh. The fibrous and/or non-woven mesh of reservoir section 320 may be hydrophobic and/or may be hydrophilic. In some non-limiting embodiments, a mesh of reservoir section 320 may include large pores to trap agglomerations through attachment to the fibers. In some non-limiting embodiments, the material of reservoir section 320 may be embedded with medicaments, such as thrombolytic drugs.

In some non-limiting embodiments, base layer 360 may be formed as a base (e.g., bottom) layer of feminine hygiene product 100. In some non-limiting embodiments, base layer 360 may be an absorbent material or a non-absorbent material. In some non-limiting embodiments, a top of base layer 360 may be in contact with a bottom of reservoir section 320. In some non-limiting embodiments, a bottom of base layer 360 may include an adhesive. For example, the adhesive at the bottom on base layer 360 may adhere to a user's underwear for positioning and stability of feminine hygiene product 100.

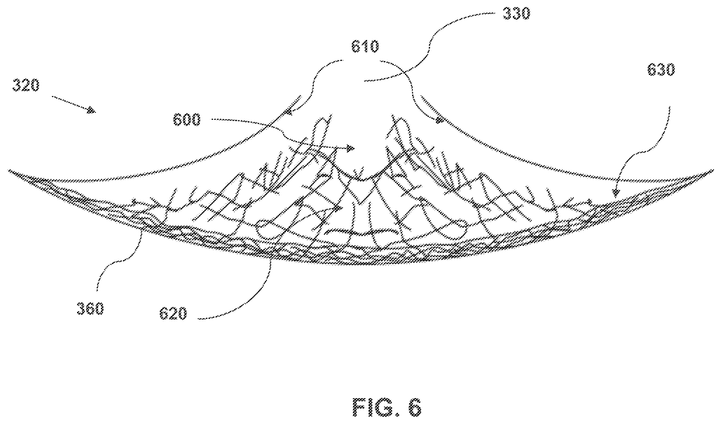

Referring now to FIG. 6, shown is a diagram of a cross section view of reservoir section 320 of feminine hygiene product 100. As shown in FIG. 6, reservoir section 320 of feminine hygiene product 100 may include cavity 600, first layer 610, second layer 620, and/or third layer 630.

In some non-limiting embodiments, reservoir section 320 may include cavity 600. For example, cavity 600 may be formed as an opening (e.g., cavity) within reservoir section 320. In some non-limiting embodiments, cavity 600 may be an opening positioned above reservoir section 320. In some non-limiting embodiments, cavity 600 may have a concave shape to increase a surface area of a material (e.g., a mesh) within cavity 600 and allow for an area to entrap agglomerations prior to breaking down into smaller particles to be retained within the network.

In some non-limiting embodiments, reservoir section 320 may include a plurality of layers. For example, reservoir section 320 may include first layer 610, second layer 620, and third layer 630. In some non-limiting embodiments, the plurality of layers of reservoir section 320 may be positioned above base layer 360. For example, first layer 610, second layer 620, and/or third layer 630 may be positioned above base layer 360. In some non-limiting embodiments, first layer 610 may be positioned above second layer 620. In some non-limiting embodiments, second layer 620 may be positioned below first layer 610 and above third layer 630. In some non-limiting embodiments, third layer 630 may be positioned below first layer 610 and second layer 620.

In some non-limiting embodiments, first layer 610 may include particulates of a medicament (e.g., a thrombolytic drug). For example, first layer 610 may be embedded with a medicament. In some non-limiting embodiments, second layer 620 may include a fibrous mesh. For example, second layer 620 may include a fibrous mesh positioned underneath (e.g., below) the particulates of the medicament in first layer 610. In some non-limiting embodiments, second layer 620 may be made of a fibrous, non-woven mesh which contains pores to trap agglomerations. In some non-limiting embodiments, second layer 620 may include a fibrous network. In some non-limiting embodiments, second layer 620 may include a pile of material. In some non-limiting embodiments, third layer 630 may be made of at least one super absorbent composite layer. For example, third layer 630 may include a plurality of absorbent composite polymers.

In some non-limiting embodiments, the plurality of layers of reservoir section 320 may include a concentration of medicament. For example, first layer 610, second layer 620, and/or third layer 630 may include a concentration of the medicament. In some non-limiting embodiments, the concentration of the medicament in first layer 610 may be higher than the concentration of medicament of the second layer and/or the concentration of medicament of the third layer.

In some non-limiting embodiments, the particulates of first layer 610 may be at a higher concentration towards distal opening 330 of reservoir section 320 and at a lower concentration distally (e.g., moving away) from distal opening 330. Additionally or alternatively, the particulates of first layer 610 may be at a higher concentration distally and at a lower concentration towards distal opening 330.

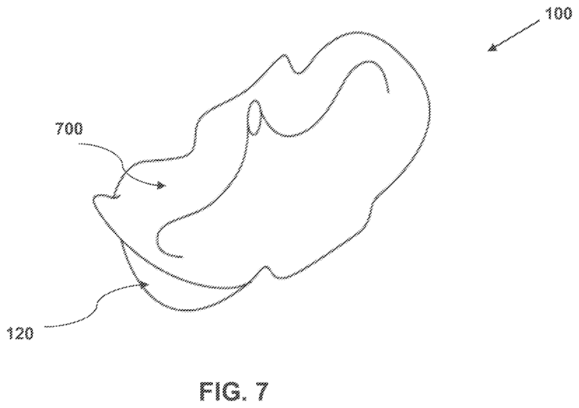

Referring now to FIG. 7, shown is a diagram of a perspective view of a non-limiting embodiment of feminine hygiene product 100 with exterior cover layer 700. In some non-limiting embodiments, exterior cover layer 700 may be positioned over top layer 120 of feminine hygiene product 100. In some non-limiting embodiments, exterior cover layer 700 may be removed before use of feminine hygiene product 100. In some non-limiting embodiments, an exothermic reaction may be triggered by removing exterior cover layer 700, resulting in exposure of a substance within feminine hygiene product 100 (e.g., particles embedded in feminine hygiene product 100) to air, and allowing for an exothermic reaction. In some non-limiting embodiments, exterior cover layer 700 may be made of any suitable packaging material. In some non-limiting embodiments, exterior cover layer 700 may include a non-breathable material.

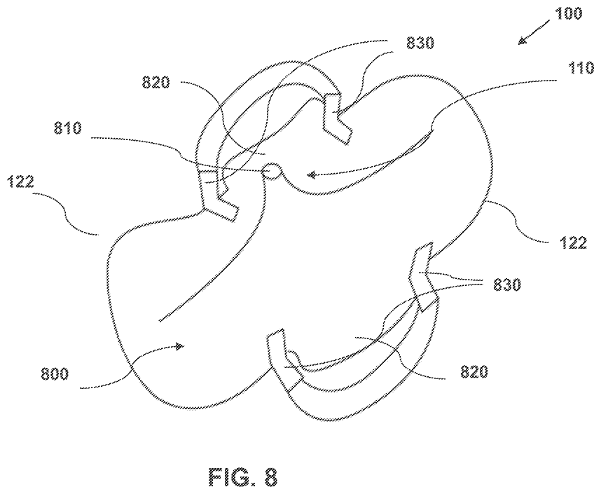

Referring now to FIG. 8, shown is a diagram of a perspective view of feminine hygiene product 100 with heating device 800. As shown in FIG. 8, heating device 800 may include aperture 810, wings 820, and/or metal strips 830. In some non-limiting embodiments, heating device 800 may be a reusable heating device or a one-time use heating device. In some non-limiting embodiments, heating device 800 may provide warmth and/or a soothing effect to the user of feminine hygiene product 100. In some non-limiting embodiments, heating device 800 may contain sodium acetate and water, which release heat upon crystallization.

In some non-limiting embodiments, heating device 800 may be sized and configured to be positioned over on top layer 120 (e.g., the same or similar to the way exterior cover layer 700 is positioned on top layer 120). In some non-limiting embodiments, heating device 800 may be attached and/or detached from feminine hygiene product 100. In some non-limiting embodiments, heating device 800 may be textured. In some non-limiting embodiments, heating device 800 may be of different thicknesses to provide support and/or comfort to the user.

In some non-limiting embodiments, heating device 800 may include aperture 810. In some non-limiting embodiments, aperture 810 is configured to fit over protrusion 110. In non-limiting embodiments, aperture 810 may have a radius. In some non-limiting embodiments, the radius of aperture 810 may be greater than the radius of protrusion 110 (FIG. 1). In some non-limiting embodiments, aperture 810 may be configured such that protrusion 110 may extend through aperture 810 for positioning within the orifice of the user or wearer.

In some non-limiting embodiments, heating device 800 may include wings 820. In some non-limiting embodiments, wings 820 may be positioned medially and facing laterally out of feminine hygiene product 100. In some non-limiting embodiments, heating device 800 may include two or more wings 820.

In some non-limiting embodiments, heating device 800 may include metal strips 830. In some non-limiting embodiments, metal strips 830 may be positioned at a junction of outer peripheral edge 122 of feminine hygiene product 100 and wings 820. In some non-limiting embodiments, metal strips 830 may be configured to be bent at one end. In some non-limiting embodiments, metal strips 830 may be configured to be bent to support a positioning of heating device 800.

In some non-limiting embodiments, wings 820 may be configured to fold such that they crease along the outer peripheral edge 122 of feminine hygiene product 100, so that they may be positioned below base layer 360 (FIG. 3) of feminine hygiene product 100. In some non-limiting embodiments, wings 820 may be affixed or fastened together after folding. In some non-limiting embodiments, folding of wings 820 may result in the bending of metal strips 830 to support positioning of heating device 800 and initiate crystallization of the chemicals therein, namely, by releasing molecules of sodium acetate crystals from metal strip 830.

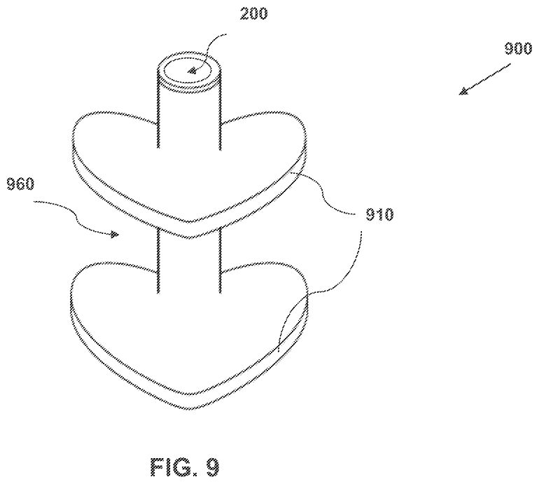

Referring now to FIGS. 9-11, FIGS. 9-11 are diagrams of perspective views of a portion of a fluid absorbing section of feminine hygiene product 100. In some non-limiting embodiments, fluid absorbing sections 900, 1000, and 1100 may include a plurality of layers 910, 1010, and 1110, respectively. In some non-limiting embodiments, plurality of layers 910, plurality of layers 1010, and/or plurality of layers 1110 may be the same as or similar to first plurality of fluid channels 310 and/or second plurality of fluid channels 312 (e.g., as shown in FIGS. 3-5). For example, plurality of layers 910, plurality of layers 1010, and/or plurality of layers 1110 may be a portion of the fluid channels of first plurality of fluid channels 310 and/or second plurality of fluid channels 312. In some non-limiting embodiments, fluid absorbing sections 900, 1000, or 1100 may include absorbing layers (e.g., first plurality of fluid channels 310 and/or second plurality of fluid channels 312) may be positioned between (e.g., sandwiched between), above, and/or below plurality of layers 910, plurality of layers 1010, or plurality of layers 1110, respectively.

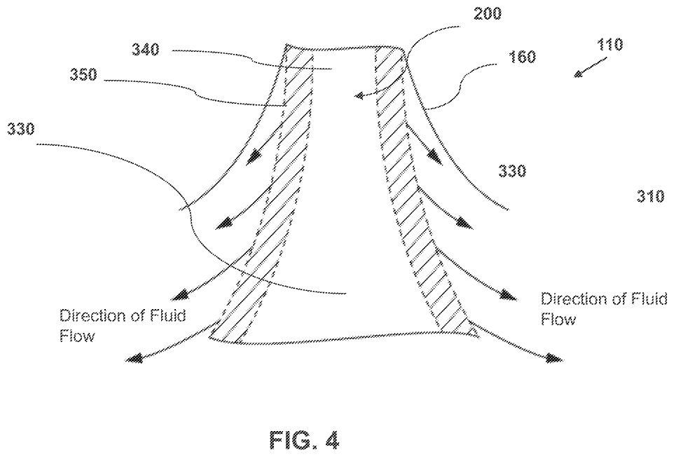

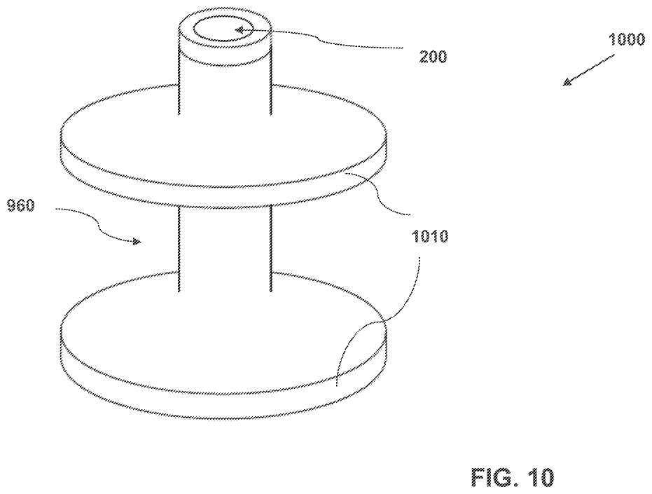

As shown in FIG. 9, the plurality of layers 910 may be formed with one or more rounded edges and/or one or more pointed edges. As further shown in FIGS. 9, primary channel 200 may include lining 960. In some non-limiting embodiments, lining 960 may be the same as or similar to primary channel lining 350 (e.g., as shown in FIGS. 3 and 4). In some non-limiting embodiments, lining 960 may be a uniform shape (e.g., a cylinder, a tube, and/or the like). In some non-limiting embodiments, lining 960 may maintain a uniform shape between each of the plurality of layers 910. As shown in FIG. 10, the plurality of layers 1010 may be formed in the shape of an ellipse.

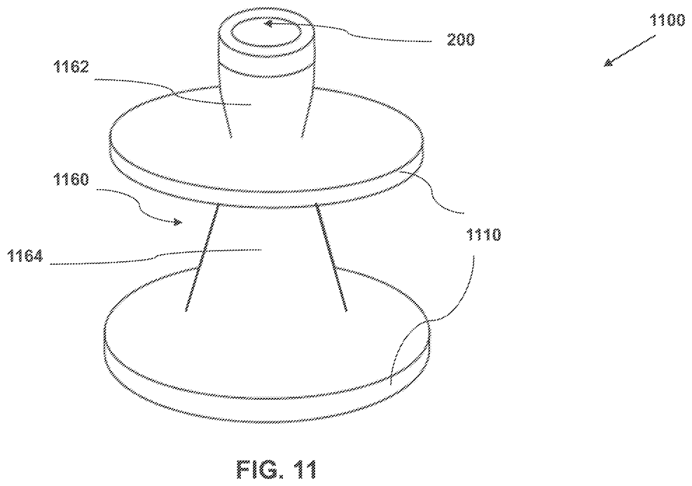

As shown in FIG. 11, plurality of layers 1110 may be formed in the shape of a circle. As further shown in FIG. 11, primary channel 200 may include lining 1160. In some non-limiting embodiments, lining 1160 may be the same as or similar to primary channel lining 350 (e.g., as shown in FIGS. 3 and 4). In some non-limiting embodiments, lining 1160 may include top portion 1162 and/or bottom portion 1164. In some non-limiting embodiments, top portion 1162 may be positioned above bottom portion 1164. In some non-limiting embodiments, top portion 1162 and/or bottom portion 1164 may vary in shape. For example, top portion 1162 may include a first portion and a second portion, where the first portion is wider than the second portion or vice versa. In some non-limiting embodiments, the first portion of top portion 1162 may be positioned above the second portion of top portion 1162. For example, a diameter of the first portion of top portion 1162 may gradually taper down to a diameter of the second portion of top portion 1162. In some non-limiting embodiments, bottom portion 1164 may include a first portion and a second portion, where the first portion is narrower than the second portion. In some non-limiting embodiments, the first portion of bottom portion 1164 may be positioned above the second portion of bottom portion 1164. In this way, fluid absorbing section 1100 allow the accommodation of agglomerations (e.g., clots, solidified fluids, tissue etc.)

Although the above methods, systems, and devices have been described in detail for the purpose of illustration based on what is currently considered to be the most practical and preferred embodiments, it is to be understood that such detail is solely for that purpose and that the present disclosure is not limited to the described embodiments or aspects but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

What is claimed is:

1. A feminine hygiene product comprising:
a top layer;
a base layer;
a protrusion formed in the top layer, the protrusion having a primary channel, wherein the protrusion is configured to be inserted within an orifice of a user;
a fluid absorbing section, wherein the fluid absorbing section comprises a plurality of fluid channels, wherein the plurality of fluid channels are configured to wick fluid away from the primary channel of the protrusion;
wherein the plurality of fluid channels have a gradient of absorbency, and a highest absorbency is distal from a proximal opening of the primary channel;
wherein the primary channel of the protrusion is positioned within the fluid absorbing section; and
a reservoir section positioned between the top layer and the base layer, wherein the reservoir section is positioned at a distal opening of the primary channel of the protrusion, and wherein the reservoir section comprises an absorbent material.

2. The feminine hygiene product of claim 1, wherein the reservoir section comprises a medicament.

3. The feminine hygiene product of claim 1, further comprising:
a longitudinal ridge formed in the top layer extending lengthwise along the base layer.

4. The feminine hygiene product of claim 1, further comprising:
an exterior cover layer configured to fit over the top layer.

5. The feminine hygiene product of claim 1, wherein the top layer is embedded with topical lotions, antibacterial lotions, or whitening agents.

6. The feminine hygiene product of claim 1, wherein an outer peripheral edge of the top layer comprises an adhesive.

7. The feminine hygiene product of claim 3, wherein an anterior crest of the longitudinal ridge comprises an absorbent material.

8. The feminine hygiene product of claim 1, wherein the top layer comprises a medicament.

9. The feminine hygiene product of claim 1, wherein the fluid absorbing section comprises a non-woven, low-swelling, absorbent polymer.

10. The feminine hygiene product of claim 1, wherein the plurality of fluid channels comprise a gradient ratio of cellulose pulp to absorbent polymer.

11. The feminine hygiene product of claim 1, wherein the protrusion of the top layer comprises a medicament.

12. The feminine hygiene product of claim 1, wherein the absorbent material of the reservoir section is porous.

13. The feminine hygiene product of claim 1, wherein the reservoir section further comprises a cavity.

14. The feminine hygiene product of claim 2, wherein the reservoir section further comprises:
a first layer comprising the medicament; and
a second layer comprising a fibrous mesh.

15. The feminine hygiene product of claim 14, wherein the reservoir section further comprises:
a third layer comprising a plurality of absorbent composite polymers.

16. The feminine hygiene product of claim 15, wherein the first layer comprises a concentration of the medicament, the second layer comprises a concentration of the medicament, and the third layer comprises a concentration of the medicament, wherein the concentration of the medicament of the first layer is higher than the concentration of the medicament of the second layer or the concentration of the medicament of the third layer.

17. The feminine hygiene product of claim 1, further comprising:
a heating device.

18. A feminine hygiene product comprising:
a top layer;
a base layer;
a protrusion formed in the top layer, the protrusion having a primary channel, wherein the protrusion is configured to be inserted within an orifice of a user;
a fluid absorbing section, wherein the fluid absorbing section comprises a plurality of fluid channels;
wherein the plurality of fluid channels are configured to wick fluid away from the primary channel of the protrusion;
wherein each of the plurality of fluid channels comprises a material having an absorption metric;
wherein the plurality of fluid channels have a gradient of absorbency;
wherein a first fluid channel of the plurality of fluid channels has a material having a first absorption metric;
wherein a second fluid channel of the plurality of fluid channels has a material having a second absorption metric;
wherein the first absorption metric is greater than the second absorption metric;
wherein the first fluid channel is adjacent to the second fluid channel; and
wherein the primary channel of the protrusion is positioned within the fluid absorbing section; and
a reservoir section positioned between the top layer and the base layer, wherein the reservoir section is positioned at a distal opening of the primary channel of the protrusion, and wherein the reservoir section comprises an absorbent material.

19. The feminine hygiene product of claim 18, wherein the reservoir section comprises a medicament.

20. The feminine hygiene product of claim 18, further comprising:
a longitudinal ridge formed in the top layer extending lengthwise along the base layer.

21. The feminine hygiene product of claim 18, further comprising:
an exterior cover layer configured to fit over the top layer.

22. The feminine hygiene product of claim 18, wherein the top layer is embedded with topical lotions, antibacterial lotions, or whitening agents.

23. The feminine hygiene product of claim 18, wherein an outer peripheral edge of the top layer comprises an adhesive.

24. The feminine hygiene product of claim 20, wherein an anterior crest of the longitudinal ridge comprises an absorbent material.

25. The feminine hygiene product of claim 18, wherein the top layer comprises a medicament.

26. The feminine hygiene product of claim 18, wherein the fluid absorbing section comprises a non-woven, low-swelling, absorbent polymer.

27. The feminine hygiene product of claim 18, wherein the plurality of fluid channels comprise a gradient ratio of cellulose pulp to absorbent polymer.

28. The feminine hygiene product of claim 18, wherein the protrusion of the top layer comprises a medicament.

29. The feminine hygiene product of claim 18, wherein the absorbent material of the reservoir section is porous.

30. The feminine hygiene product of claim 18, wherein the reservoir section further comprises a cavity.

31. The feminine hygiene product of claim 18, wherein the reservoir section further comprises:
a first layer comprising a medicament; and
a second layer comprising a fibrous mesh.

32. The feminine hygiene product of claim 31, wherein the reservoir section further comprises:
a third layer comprising a plurality of absorbent composite polymers.

33. The feminine hygiene product of claim 32, wherein the first layer comprises a concentration of the medicament, the second layer comprises a concentration of the medicament, and the third layer comprises a concentration of the medicament, wherein the concentration of the medicament of the first layer is higher than the concentration of the medicament of the second layer or the concentration of the medicament of the third layer.

34. The feminine hygiene product of claim 18, further comprising:
a heating device.

35. A feminine hygiene product comprising:
a top layer;
a base layer;
a protrusion formed in the top layer, the protrusion having a primary channel, wherein the protrusion is configured to be inserted within an orifice of a user;
a fluid absorbing section, wherein the fluid absorbing section comprises a plurality of fluid channels, wherein the plurality of fluid channels are configured to wick fluid away from the primary channel of the protrusion;
a reservoir section positioned between the top layer and the base layer, wherein the reservoir section is positioned at a distal opening of the primary channel of the protrusion, and wherein the reservoir section comprises an absorbent material; and
a heating device, wherein the heating device comprises an aperture, and wherein the aperture is configured to fit over the protrusion formed in the top layer.

36. The feminine hygiene product of claim 35, wherein the reservoir section comprises a medicament.

37. The feminine hygiene product of claim 35, further comprising:
a longitudinal ridge formed in the top layer extending lengthwise along the base layer.

38. The feminine hygiene product of claim 35, wherein an outer peripheral edge of the top layer comprises an adhesive.

39. The feminine hygiene product of claim 37, wherein an anterior crest of the longitudinal ridge comprises an absorbent material.

40. The feminine hygiene product of claim 35, wherein the fluid absorbing section comprises a non-woven, low-swelling, absorbent polymer.

41. The feminine hygiene product of claim 35, wherein the plurality of fluid channels comprise a gradient ratio of cellulose pulp to absorbent polymer.

42. The feminine hygiene product of claim 35, wherein the protrusion of the top layer comprises a medicament.

43. The feminine hygiene product of claim 35, wherein the absorbent material of the reservoir section is porous.

44. The feminine hygiene product of claim 35, wherein the reservoir section further comprises a cavity.

45. The feminine hygiene product of claim 36, wherein the reservoir section further comprises:
a first layer comprising the medicament; and
a second layer comprising a fibrous mesh.

46. The feminine hygiene product of claim 45, wherein the reservoir section further comprises:
a third layer comprising a plurality of absorbent composite polymers.

47. The feminine hygiene product of claim 46, wherein the first layer comprises a concentration of the medicament, the second layer comprises a concentration of the medicament, and the third layer comprises a concentration of the medicament, wherein the concentration of the medicament of the first layer is higher than the concentration of the medicament of the second layer or the concentration of the medicament of the third layer.

48. The feminine hygiene product of claim 35, wherein the heating device comprises two wings, wherein each of the two wings comprises a metal strip, and wherein the metal strip is configured to be bent to support a positioning of the heating device.

49. The feminine hygiene product of claim 48, wherein the two wings are configured to be fastened together.

50. The feminine hygiene product of claim 35, wherein the plurality of fluid channels have a gradient of absorbency, and a highest absorbency is distal from a proximal opening of the primary channel; and
wherein the primary channel of the protrusion is positioned within the fluid absorbing section.
